(19) United States
(12) Patent Application Publication
(10) Pub. No.: US 2006/0105063 A1
(43) Pub. Date: May 18, 2006

(54) Synergic combination of compositions containing aloe vera isolates and their therapeutic application

(75) Inventors: Kent Hann, Minnetonka, MN (US); Luke Lentz, Destin, FL (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: Kent Hann

(21) Appl. No.: 11/281,314

(22) Filed: Nov. 17, 2005

Related U.S. Application Data
(60) Provisional application No. 60/628,594, filed on Nov. 18, 2004.

(51) Int. Cl. A61K 36/886; A61K 31/715

(52) U.S. Cl. 424/744; 514/54

(57) ABSTRACT

Synergic compositions containing an Aloe vera isolate and optionally, a prebiotic, Fucose, and/or other treatment regimens, as well as methods for their preparation and use in treating disease conditions such as neurological syndromes, chronic pain illnesses, inflammatory bowel diseases, and viral diseases are disclosed.

### RELATED APPLICATIONS

This application claims priority to U.S. Provisional Patent Application Ser. No. 60/628,594, titled Synergic Combination of Pre-biotics, Aloe Vera Isolates and Their Therapeutic Applications, filed Nov. 18, 2004, the contents of which are hereby incorporated by reference.

### FIELD OF INVENTION

The present invention relates to compositions and methods for treating disease conditions such as neurological syndromes, chronic pain illnesses, inflammatory bowel diseases, and viral diseases.

### BACKGROUND

The genus Aloe has over the years proved to be an important source of biologically active compounds. Aloe vera, a member of the lily family, is generally recognized as the “True Aloe” because of its wide use in folk medicine and reportedly effective healing power.

“Aloes”, the dried juice which flows from the transversely cut bases of the large leaves, have been used for the treatment of disease, particularly in connection with the digestive system. The mucilaginous gel obtained from the leaves is well reputed for its therapeutic effects, and its beneficial effects on skin diseases and wound-healing, and its anti-inflammatory activity are well documented.

During the last decade of the twentieth century new medical uses of Aloe vera's gel and its derivatives were investigated. These experimental therapies, addressing mainly allergies, immuno suppression, autoimmune disorders, cancer, and viral diseases, are mostly rooted in the gel's components or their derivative's capacity to enhance both humoral (molecular) and cellular immunity.

Further, there is limited evidence available on the value of Aloe vera's gel and its derivatives in treating viral common colds, alveolar osteitis, oral ulcers/aphthous stomatitis, eye ulcerations/viral keratitis, herpes, chronic fatigue syndrome/Epstein-Barr virus, cytomegalovirus infection as a result of HIV infection, fungal infections associated with HIV, cryptosporidiosis associated with HIV, resistant human tuberculosis associated with HIV resistant avian tuberculosis in humans associated with HIV, pneumocystis carinii infection (pneumonia) associated with HIV, Alzheimer's disease, hyperthyroidism, multiple sclerosis, symptoms associated with cystic fibrosis, sequelae to a rheumatic fever episode, depression/anxiety, cuts and scratches, tic douloureux/trigeminal neuralgia, dry eye syndrome, cataract, peptic ulcer, diarrhea, malabsorption syndrome, inflammatory bowel disease/ulcerative colitis/Crohn's disease/spastic enterocolitis, irritable bowel syndrome, hypercholesterolemia, diabetes, and arthritis.

Without intending to be bound by theory, there are several possibilities as to the chemical entity responsible for a given therapeutic effect of the Aloe vera. For example, the identity of the active substance(s) in Aloe vera gel has been postulated to be: i).—a glucomannan containing a small proportion of glucuronic acid residues, of molecular weight 420,000-520,000, ii).—an arabinogalactoglucomannan (“Aloeride”/NP18298) of molecular weight greater than 2 million, iii).—a long-chain acetylated galactomannan, (“Carrisyn”/“Aloe polymannose/AVMP/Manapol) iv).—a linear acetylated mannan (i.e., a linear acemannan), v).—an acetylated glucogalactomannan, vi).—two arabinoglucogalactomannans, with molecular weights of 200,000 and 320,000, vii).—a polysaccharide derived from mannose, glucose, arabinose, rhamnose and galactose “including nitrogen, sulfur and phosphorus” and having a molecular weight of about 70,000 (“Aloeferon”) viii).—a mixture of polysaccharides, ix).—two high-molecular weight glycoproteins (Aloctin A and Aloctin B) plus an unspecified number of low molecular weight components, x).—a hemagglutinating glycoprotein (Aloctin A) of molecular weight about 18,000, xi).—a glycoprotein of molecular weight 29,000, xii).—a glycopeptide of molecular weight 5,500, xiii).—three lectins (hemagglutinating glycoproteins), xiv).—“glycoproteins or a mixture of polysaccharides and proteins (Aa-50)”, xv).—a glycoprotein “Alprogen”/G1G1M1DI2” with an apparent molecular weight of 5,500, xvi).—a protein (ATF1011), xvii).—an enzyme (a carboxypeptidase), xviii).—an enzyme (a peroxidase), xix).—gibberellins, xx).—a number of “bioactive factors”, and xxi).—beta-sitosterol.

In related developments, Akev and Can in Akev, N.; Can, A. Phytother. Res. 13, 489-493(1999), reported the isolation of two hemagglutinating glycoproteins, Mandal et al. obtained a galactan in Mandal, G. et al. Carb. Res. 86, 247-257 (1980), and a pectic acid in Mandal, G. et al. Indian J. Chem., Sect B, 22B, 890-893(1983), Ni et al. in Ni, Yawei et al. PCT Int. Appl. WO 99 58575, extracted several pectins, Haq and Hannan in Haq, Q. N.; Hannan, A. Bangladesh J. Sci. Ind. Res. 16, 68-72(1981), extracted a linear glucogalactomannan using aqueous acetone, and Gowda et al., Gowda, D. C. et al. Carbohydr. Res. 72, 201-205(1979), found that “the gel from the leaves of Aloe vera is composed of at least four different partially acetylated linear glucomannans that differ in their glucose-to-mannose ratios and acetyl contents”.

Furthermore, oligosaccharides/polysaccharides with high biological activity can be obtained by controlled cleavage (using cellulase enzymes, for instance) of native precursor “block polysaccharides” or “structural polysaccharides”. Thus, Strickland et al. in Strickland, F. M. et al. J. Invest. Dermatol. 102, 197-204 (1994), and Strickland, Faith M. et al. PCT Int. Appl. WO 98 09,635, obtained a highly active oligosaccharide (“containing about 75% glucose, about 25% mannose and trace galactose”) of molecular weight 1,000-5,000 from a native precursor polysaccharide of molecular weight greater than 2,000,000; Qiu and Mahiou in Qiu, Zhihua; Mahiou, Belaid. PCT Int. Appl. WO 99 19,505, and Qiu et al., Qiu, Z. et al. Planta Med. 66, 152-156(2000), claim that “the resulting immunomodulatory complex (“Immuno-10”, of average molecular weight 80,000) has a higher activity and is more stable than bulk carbohydrates isolated using prior art precipitation schemes”, and both Avalos and Danhof in Avalos, Ramiro Estrada; Danhof, Ivan E. U.S. Pat. No. 6,083,508 4 Jul. 2000, and Coats in Coats, Billy C. U.S. Pat. No. 5,356,811; 18 Oct., 1994, advocate cellulase treatment, as well as Kobayashi et al. in Kobayashi, Mitsuhiro et al. JP 60109526; 15 Jun. 1985, who pioneered such treatment. Additionally, Qiu et al. in Qiu, Z. et al. Planta Med. 66, 152-156(2000), have stated that whereas partially digested Aloe barbadensis polysaccharides activate macrophages, native Aloe barbadensis gel has no effect.

It should be noted that plausible explanations of most of the compositional variations reported for Aloe Vera carbohydrates have been offered by Yaron in Yaron, A. Phytother. Res., 1993, 7 (Spec. issue: Proceedings of the International Congress of Phytotherapy, 1991), S11-S13, Strickland et al. in Strickland, Faith M. et al. PCT Int. Appl. WO 98 09,635, and most recently by Femenia et al. in Femenia, A. et al. Carbohydr. Polym. 39, 109-117(1999), that Okyar et al. in Okyar, A. et al. Phytother. Res. 15, 157-161 (2001), found the pulp of Aloe vera leaves-devoid of the gel-useful in the treatment of non-insulin mediated diabetes mellitus in rats, that Taylor in Taylor, Allan. PCT Int. Appl. WO 00 50056, advocates the use of Aloe vera fibers for regulating lower bowel function/irritable bowel syndrome and that—according to Davis, Davis, Robert H. U.S. Pat. No. 5,487,899; 30 Jan. 1996,—Aloe vera fibers contribute to wound healing when applied topically.

Prebiotics have also been shown to have beneficial effects. Generally, a “prebiotic” may be defined as a non-digestible food ingredient that beneficially affects the host by selectively stimulating the growth and/or the activity of one or a limited number of bacterial strains in the colon. Inulin-type fructans are part of the “dietary fiber complex” and they belong in the categories of non-absorbable/non-digestible/digestion-resistant carbohydrates of non-starch polysaccharides and of “prebiotics.” Inulin-type fructans are complex carbohydrates whose molecules comprise chains of beta-D-fructofuranose units coupled by beta (2→1) linkages and terminated by either a beta-D-glucopyranosyl or a beta-D-fructofuranosyl residue. They constitute a group of oligosaccharides/polysaccharides that occur as storage carbohydrates in many vegetable sources that include common foodstuffs such as wheat, onions, asparagus, artichoke and bananas. Currently, food components that seem to exert the best prebiotic effects are inulin-type fructans. Inulin [CASRN 9005-80-5] and oligofructose are the most important and common inulin-type fructans; both inulin and oligofructose are commercially extracted from chicory (Cichorium intybus) roots.

It is believed that the major nutritional and physiological effects of a model prebiotic such as inulin concern the composition of the colonic microflora, the gastrointestinal physiology, the immune function, the bioavailability of calcium and other minerals, lipid metabolism and colonic carcinogenesis. Scientific evidence for a bifidogenic effect of inulin supports the claim that this prebiotic effectively modifies the composition of the colonic microflora of experimental animals and human beings; an indirect consequence of the stimulation of bifidobacterial growth is fecal bulking/improved bowel function, for which convincing evidence exists.

Inulin has been studied in a variety of contexts. For example, PCT Int. Appl.WO 01 15,714 and PCT Int. Appl. WO 01 64,225 discusses inulin-based compositions useful for enhancing general immunity, cancer-inhibiting activity in animal models. In addition, several patents, such as Fukuzaki, E. et al, JP 02,172,921; 4 Jul. 1990, Roberfroid, M. et al, EP 692,252; 17 Jan. 1996, Van Loo, Jan; Frippiat, Anne, EP 879,600; 25 Nov. 1998, Taper, Henryk et al, EP958,825; 18 May 1998, and Kim, Hyung Min, KR 2000 2,087; 15 Jan. 2000, discuss compositions for cancer prevention/treatment based on inulin; again, their main focus is colon cancer and/or cancer in non-ruminant mammals. Inulin's use for treating non-insulin dependent diabetes has been considered; and at least three patents, Von Sonnleithner, F. M., DE 4,442,975; 5 Jun. 1996, Jaussan, Veronique et al, JP 11,18,725; 26 Jan. 1999, and Nakayama, Shigeo, JP 2002 00,216; 8 Jan. 2002, covering diabetes treatment have been issued.

### SUMMARY OF THE INVENTION

Embodiments of the invention relate to a method and composition useful for treating disease conditions such as neurological syndromes (e.g., reflex sympathetic dystrophy), chronic pain illnesses (e.g., fibromyalgia), inflammatory bowel diseases (e.g., Crohn's disease), and viral diseases (e.g., HIV/AIDS). This method and system comprises the delivery of a composition containing an Aloe vera isolate and one or more of a prebiotic (e.g., an inulin-type fructan) and other therapy regimens to a mammal in need thereof. In some embodiments, the composition further includes Fucose.

In some embodiments, the composition includes a pharmaceutical composition comprising the above-mentioned components and one or more pharmaceutically acceptable excipients, carriers, diluents, or adjuvants. This invention further relates to a method for preparing pharmaceutically acceptable dosage forms containing the aforementioned active principles and-optionally-one or more pharmaceutically acceptable excipients, carriers, diluents or adjuvants, such dosage forms being capable of releasing the active ingredients in a mammal in need thereof. Further, the invention also comprises the use of combination therapies involving administration of the aforementioned active ingredients to, for example, humans and/or non-human mammals, i.e. as veterinary medication for treatment of said non-human mammals in need thereof.

In other embodiments, the invention includes methods of treating a disease condition, comprising delivering a composition containing an Aloe vera isolate derived from total process aloe to a mammal in need thereof along with other therapeutic regimens.

Accordingly, methods and compositions in accordance with embodiments of the invention provide an effective and inexpensive treatment option for several disease conditions.

### BRIEF DESCRIPTION OF THE DRAWING

FIG. 1 shows a bar graph of the percent change in CD4 count by patient as described in Examples 7-10.

FIG. 2 shows a graph depicting the change in CD4 count by time as described in Example 12.

FIG. 3 shows a graph depicting the change in CD4 count by patient as described in Example 12.

### DESCRIPTION OF THE INVENTION

Embodiments of the invention include methods and compositions useful for treating disease conditions such as neurological syndromes (e.g., reflex sympathetic dystrophy), chronic pain illnesses (e.g., fibromyalgia), inflammatory bowel diseases (e.g., Crohn's disease), and viral diseases (e.g., HIV/AIDS). In some embodiments, this system comprises the delivery of therapeutically effective amounts of a composition comprising an Aloe vera isolate and one or more of a prebiotic (e.g., inulin or oligofructose) and other therapeutic regimens to a mammal (e.g., to a mammal's gut).

The Aloe vera component of the invention may be derived from any suitable source and/or process, including traditional hand-filleted aloe process, whole leaf aloe process, powdered forms of aloe utilizing either spray-dried aloe powder, lyophilized aloe powder or dehydrated aloe powder, and Total Process Aloe (TPA). Certain desirable embodiments of the invention utilize Aloe vera derived from TPA because it is believed that process leaves more of the desirable isolates such as long chain polysaccharides, solids, calcium, magnesium, and malic acid in the product.

An example of TPA is described in U.S. Pat. No. 6,083,508, the relevant contents of which are hereby incorporated by reference. In that example, the aloe leaves are hand-filleted by the traditional intensive method. Instead of being discarded, the green rinds and the mucilage layer that are generated in the filleting process are utilized as part of the new process to provide the aloe product that has the high concentration of desirable constituents. Thus the use of the discarded residue in the hand-filleted process in combination with a new process recovers the highest concentration of potentially beneficial aloe constituents found in the mucilage and the green rinds where the constituents are synthesized by the vascular bundle cells powered by energy developed in the green chlorophyll-containing rind cells through sun-induced photosynthesis. Accordingly, TPA may comprise an aloe product derived from aloe leaves comprising the steps of filleting each aloe leaf to obtain both a fillet and leaf residue, grinding only the leaf residue (rind portions and mucilage) into a slurry, and preparing an aloe product from the slurry. In some circumstances, cellulase is added to the slurry to digest solids by an enzymatic action and the slurry with the cellulase is continuously stirred for a period of time in the range of from about two to about four hours to obtain maximum digestion of the cellulase-reinforced hexagons in the slurry. TPA useful in some embodiments of the invention may be commercially obtained from sources such as, Improve U.S.A., Inc., 215 Dalton Drive, Suite D, DeSoto, Tex. 75115.

Some embodiments of the invention include a prebiotic. The prebiotic may include any compound able to provide the desired synergistic effect. In some embodiments, the prebiotic comprises a fructan. Fructans are polymers of fructose. Fructans have a general structure of a glucose linked to multiple fructose units. There are several types of fructans present in nature. These types are distinguished on the basis of the glycosidic linkages by which the fructose residues are linked to each other. They can broadly be divided into 3 groups. The first group are the inulins, which are linear fructans, where the fructose units are (mostly) linked via a beta (2->1) bond. For example, the inulin-type fructans may comprise chains of beta-D-fructofuranose units coupled by beta (2→1) linkages and terminated by either a beta-D-glucopyranosyl or a beta-D-fructofuranosyl residue. Such an inulin-type fructan may be derived from a chicory root. Inulin type fructan useful in some embodiments of the invention may be commercially obtained from sources such as Cargill, Inc. PO Box 9300, Minneapolis, Minn. The structure shown below as (a) is 1-kestose. This is the shortest fructan of the inulin type.

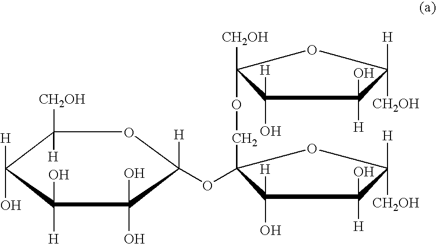

The second group are the levans, which are also linear fructans, but where the fructose units are (mostly) linked via a beta(2->6) bond. This type of fructan is found in a large part of the monocotyledons and in almost all bacterial fructans. The structure shown as (b) below is 6-kestose. This is the shortest fructan of the levan type.

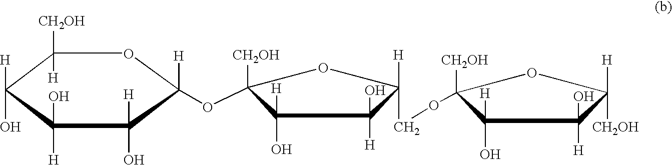

The third group are the fructans of the mixed type, which are also referred to as the graminan type. These fructans have both beta(2->1) and beta(2->6) linkage bonds between the fructose units, and thus contain branches. These fructans are found in grasses, for example. The structure shown as (c) below is 6,6&1 kestopentaose. This is one of the simplest fructans of this type.

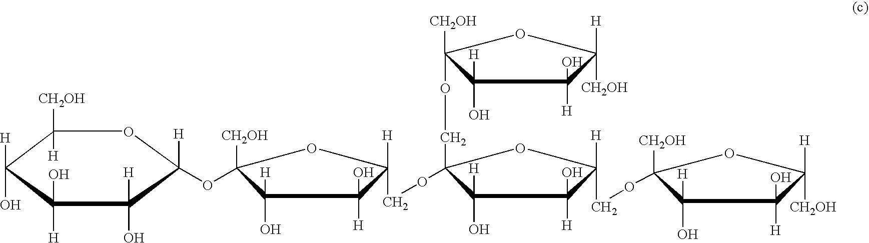

In some embodiments, the composition may also contain Fucose. Fucose is one of eight essential sugars the body requires for optimal function of cell-to-cell communication. The L form is the common form of the sugar, while the D form is a synthetic galactose analogue. Fucose may be found in several medicinal mushrooms, seaweeds as kelp and wakame, and beer yeast. Fucose should not be confused with Fructose, which is a monosaccharide found in fruits and honey.

When taken orally, Fucose is readily absorbed from the small intestine and incorporated either directly or after metabolism into glycoproteins. Unabsorbed Fucose is metabolized by friendly intestinal bacteria. Studies have shown that, when Fucose is given in extraordinarily high amounts, there are little to no side effects. The only related oral toxicity that has been found was from animals ingesting a diet composed of 20% Fucose. This amount appeared to reduce nerve conduction velocity as well as collagen production. What similar effects would be in humans has yet to be determined. However, microscopic examination of the liver, kidney, pancreas, and the sternal bone marrow of Fucose-treated rats revealed no abnormalities. According to available studies, it appears that oral doses as great as 34 grams in a healthy 150-pound adult is considered safe. Maximum blood levels would be expected one hour after ingestion and would be eliminated from the bloodstream eight to twelve hours later. Therefore, twice daily doses of any amount are recommended to maintain sufficient blood levels. In humans, Fucose is excreted mainly in the urine at a rate of approximately 17 micrograms per minute.

Fucose concentrations are found in such areas as: a) at the junctions between nerves, implying that a deficiency could affect synaptic transmissions; b) in the proximal tubules of the human kidney, indicating the need for this saccharide for proper kidney function; c) in the testes, suggesting that it plays an important role in reproduction; and d) in the outer layer of skin, where it may be involved in maintaining skin hydration.

Fucose metabolism appears to be altered in various diseases. Several studies have concluded that Fucose metabolism is abnormal in those with cystic fibrosis, diabetes, and during episodes of shingles, which is caused by a herpes virus. These studies go on to suggest that the sugar is active against other herpes viruses. In addition, the saccharide guards against respiratory tract infections and inhibits allergic reactions. Liver function and serum protein levels were also affected by a deficiency of Fucose. Levels of Fucose are also low in those with rheumatoid arthritis, and supplementation has shown promise as an effective treatment.

Other studies have shown Fucose can be incorporated into certain areas of the body where and when it is most needed. For instance, Fucose incorporated into the photoreceptor layer of the retina, may help with the biosynthesis of rod cell glycoproteins. In psoriasis, Fucose may play a significant role in the disease process because of altered glycoprotein distribution. Normally, skin keratinocytes and non-psoriatic cells have most of their Fucose on the plasma membrane, whereas psoriatic cells retain most of their Fucose within the cytoplasm.

Fucose is believed to have several functions. For example, Fucose glycoconjugates (glycoproteins and glycolipids) may be an essential part of eliminating or reversing such disease processes as cancer, inflammation, and immunity. Further, Fucose may be important for efficient neuron transmission in the brain. According to studies, Fucose is known to influence brain development and may also help improve the brain's ability to create long-term memories. Several studies have shown that, by inhibiting the Fucose-containing protein, amnesia developed. In addition, Fucose is a powerful immune modulator. It is distributed in macrophages, which are important to immune function. There have been numerous well-documented benefits for its necessity in immune function, especially that of an overactive immune system, the cause of autoimmune disorders. Fucose is showing promise in its ability to normalize immune function. Fucose is particularly active in inflammatory diseases and appears to have the ability to suppress such allergic skin reactions as contact dermatitis.

In addition, Fucose appears to be a possible treatment for cancers such as breast cancer. U-fucoidan, a complex polysaccharide found in brown seaweed, was able to kill cancer cells in vitro in lab animals within 72 hours. Interestingly, the destruction was self-induced (apoptosis), suggesting that the sugars were able to break down the DNA within each cancer cell through enzyme action. Fucose useful for some embodiments of the invention may be commercially obtained from sources such as Spectrum Chemicals & Laboratory Products, 14422 South San Pedro Street, Gardena, Calif. 90248, USA.

As described above, embodiments of the invention include compositions comprising Aloe vera, a prebiotic, and Fucose. It should be noted that the therapeutic effects of such combinations do not correspond to the sum of the individual components' effects. That is, the methods and compositions described herein are synergic, with potentiation of the healing effect of one ingredient by the other. This mutual healing potentiation is especially evident in the treatment of viral diseases. For example, it is known that the bifidogenic/immunostimulating effect of inulin or oligofructose in human beings requires administering at least 2-4 grams per day (see Gibson, G. R.; Br. J. Nutr. 80, (suppl 2) S209-S212 (1998), and Roberfroid, M. B.; Br. J. Nutr. 80, (suppl 2) S197-S202 (1998)), which is an amount substantially higher than that involved in most of the examples described below.

The compositions and methods described herein may be used to treat disease conditions in any mammal. For example, such compositions and methods may be used to treat a disease condition in a human. Such compositions and methods may also be used in non-human mammals (e.g., canine, feline, equestrian) in veterinary applications.

Disease conditions that may be treated with the compositions and methods described herein include neurological syndromes (e.g., reflex sympathetic dystrophy), chronic pain illnesses (e.g., fibromyalgia), inflammatory bowel diseases (e.g., Crohn's disease), and viral diseases (e.g., HIV/AIDS).

Reflex Sympathetic Dystrophy Syndrome (RSD)—also known as Complex Regional Pain Syndrome (CRPS) is a chronic neurological syndrome characterized by severe burning pain, pathological changes in bone and skin, excessive sweating, tissue swelling, and extreme sensitivity to touch. Generally, there are two types of RSD, Type I and Type II. Type I includes cases in which the nerve injury cannot be immediately identified. Type II includes cases in which a distinct “major” nerve injury has occurred. RSD/CRPS may be described in terms of an injury to a nerve or soft tissue (e.g. broken bone) that does not follow the normal healing path, and its development does not appear to depend on the magnitude of the injury. The sympathetic nervous system seems to assume an abnormal function after an injury Since there is no single laboratory test to diagnose RSD/CRPS, the physician must assess and document both subjective complaints (medical history) and, if present, objective findings (physical examination).

Fibromyalgia (FM) is an increasingly recognized chronic pain illness which is characterized by widespread musculoskeletal aches, pain and stiffness, soft tissue tenderness, general fatigue and sleep disturbances. The most common sites of pain include the neck, back, shoulders, pelvic girdle and hands, but any body part can be involved. Fibromyalgia patients experience a range of symptoms of varying intensities that wax and wane over time. It is estimated that approximately 3-6% of the U.S. population has FM. Although a higher percentage of women are affected, it does strike men, women and children of all ages and races. Currently there are no laboratory tests available for diagnosing Fibromyalgia. Doctors must rely on patient histories, self-reported symptoms, a physical examination and an accurate manual tender point examination.

Crohn's disease (also called ileitis or enteritis) is an inflammatory bowel disease (IBD), the general name for diseases that cause inflammation in the intestines. Crohn's disease causes inflammation in the small intestine. Crohn's disease usually occurs in the lower part of the small intestine, called the ileum, but it can affect any part of the digestive tract, from the mouth to the anus. The inflammation extends deep into the lining of the affected organ. The inflammation can cause pain and can make the intestines empty frequently, resulting in diarrhea. Crohn's disease can be difficult to diagnose because its symptoms are similar to other intestinal disorders such as irritable bowel syndrome and to another type of IBD called ulcerative colitis. Ulcerative colitis causes inflammation and ulcers in the top layer of the lining of the large intestine. Crohn's disease affects men and women equally and seems to run in some families. About 20 percent of people with Crohn's disease have a blood relative with some form of IBD, most often a brother or sister and sometimes a parent or child.

Acquired Immune Deficiency Syndrome (AIDS) is caused by a virus called HIV, the Human Immunodeficiency Virus. The body will try to fight the infection by the production of antibodies. If the antibodies are present in blood, that indicates a HIV infection. In the United States, there are about 800,000 to 900,000 people who are HIV-positive. Over 300,000 people are living with AIDS. Each year, there are about 40,000 new infections.

Being HIV-positive, or having HIV disease, is not the same as having AIDS. Many people are HIV-positive but do not get sick for many years. As HIV disease continues, it slowly wears down the immune system. Viruses, parasites, fungi and bacteria that usually don't cause any problems can cause sickness if the immune system is damaged. These are called “opportunistic infections”

After infection by HIV, some people get fever, headache, sore muscles and joints, stomach ache, swollen lymph glands, or a skin rash for one or two weeks. Most people think it's the flu. Some people have no symptoms. The virus will multiply in the body for a few weeks or even months before the immune system responds. After the first flu-like symptoms, some people with HIV stay healthy for ten years or longer. But during this time, HIV is damaging the immune system.

One way to measure the damage to the immune system is to count CD4+ cells. These cells, also called “T-helper” cells, are an important part of the immune system. Healthy people generally have between about 500 and about 1,500 CD4+ cells per milliliter of blood. Without treatment, CD4+ cell counts will most likely go down in individual with HIV. Further, signs of HIV disease, like fevers, night sweats, diarrhea, or swollen lymph nodes, may develop.

HIV disease becomes AIDS when the immune system is seriously damaged. Generally, under some definitions, if less than 200 CD4+ cells are present, or if your CD4+ percentage is less than 14%, the disease has progressed to AIDS.

Having an opportunistic infection can also be a sign of AIDS. The most common opportunistic infections are PCP (Pneumocystis pneumonia), a lung infection; KS (Kaposi's sarcoma), a skin cancer; CMV (Cytomegalovirus), an infection that usually affects the eyes; and Candida, a fungal infection that can cause thrush (a white film in your mouth) or infections in your throat or vagina. AIDS-related diseases also includes serious weight loss, brain tumors, and other health problems.

The relative concentrations and dosage levels of the composition components to treat disease conditions such as those described above may comprise any ratio and level suitable for the desired effect. The quantity of the composition to be administered will be determined on an individual basis, and will be based at least in part on consideration of the severity of infection or injury in the patient, the patient's condition or overall health, the patient's weight, the time available before other treatment and the means of administration (e.g. a larger amount may be administered for oral compositions than for systemic compositions).

The dosage forms should be capable of releasing the active ingredients in the mammal (e.g., in the gut of a mammal). The preferred dosage levels are about 100 mg. TPA isolate plus 150 mg inulin-type fructan per 12 Kg of patient (or non-human mammal) weight per day but—as discussed above-should be adjusted on an individual basis, and may be increased by a factor of up to about ten or decreased by a factor of up to about five if deemed necessary. Functionally, the limiting factor may be the individual patient's tolerance to potential adverse gastrointestinal symptoms (gases and bloating) caused by inulin/oligofructose in human subjects consuming over about 15 g per day, Williams, C. M.; Jackson, K. G., Br. J. Nutr. 87, (suppl 2) S261-S264 (2002).

With regard to relative concentrations of the components, in some embodiments, the Aloe vera may comprise about 30-70% of the composition by weight and the prebiotic may comprise about 30-70% of the composition by weight. Optionally, in some embodiments, the Aloe vera may comprise about 40-60% of the composition by weight and the prebiotic may comprise about 40-60% of the composition by weight. In yet other embodiments, the Aloe vera may comprise about 45-55% of the composition by weight and the prebiotic may comprise about 45-55% of the composition by weight.

In embodiments of the invention containing Fucose, the amount and relative concentrations of the composition components may comprise any ratio suitable for the desired effect. In some embodiments, the Aloe vera may be about 30-70% of the composition by weight. Further, in such embodiments, the prebiotic may be about 30-70% of the composition by weight. In addition, the Fucose may be about 10-50% (e.g., about 25%) of the composition by weight.

The compositions described herein may be administered in any suitable manner (e.g., oral, enteral, topical, parenteral, intravenous, subcutaneous, intraperitoneal, intramuscular, or intranasal). For example, the composition may be administered orally, in an encapsulated powder and/or as a reconstituted liquid. Further, the various components need not be administered in the same form and/or manner. For example, the Aloe vera component may be taken in a pill form and the inulin may be taken separately in a powder form.

The composition administered in the methods of the present invention can optionally include other components such as excipients, carriers, diluents, adjuvants and/or other beneficial active components. In such embodiments, the dosage amounts and relative concentration percentages discussed above do not include such other components. Other components included in a particular composition may be determined primarily by the manner in which the composition is to be administered. For example, a composition to be administered orally in tablet form can include, fillers (e.g. lactose), binders (e.g., carboxymetyl cellulose, gum Arabic, gelatin), adjuvants, emulsifying agents, flavoring agents, coloring agents, drying agents, other active agents (e.g. pharmaceuticals, minerals, vitamins) and coating materials (e.g., wax or plasticizer).

Embodiments having the components provided in pill form may comprise any suitable dosage amount and/or relative component concentrations. For example, a pill in accordance with some embodiments of the invention may be about 300 mg to about 700 mg. Optionally, a pill in accordance with some embodiments of the invention may be about 400 mg to about 700 mg. Further optionally, a pill in accordance with some embodiments of the invention may be about 500 mg. In some embodiments, such pills may comprise about 200 mg to about 400 mg Aloe vera extract, about 150 mg to about 250 mg inulin, and about 5 mg to about 50 mg Fucose. The pill may also comprise, in some embodiments, less than about 1% other materials, such as, for example, drying agents.

Further, in some embodiments, the compositions described herein may be administered along with other therapeutic regimens, such as antiviral drug therapy regimens and antibiotics. Examples of suitable antiviral drug therapy regimens, particularly in the context of HIV/AIDS treatment, include Fusion Inhibitors such as Enfuvirtide (Fuzeon, T-20); Normucleoside Reverse Transcriptase Inhibitors (NNRTIs) such as Delavirdine (Rescriptor), Efavirenz (Sustiva), and Nevirapine (Viramune); Nucleoside Reverse Transcriptase Inhibitors (NRTIs) such as Abacavir (Ziagen), Abacavir+Lamivudine (Epzicom), Abacavir+Lamivudine+Zidovudine (Trizivir), Didanosine (Videx, ddI), Emtricitabine (Emtriva, FTC), Emtricitabine+Tenofovir DF (Truvada), Lamivudine (Epivir, 3TC), Lamivudine+Zidovudine (Combivir), Stavudine (Zerit, d4T), Tenofovir DF (Viread), Zalcitabine (Hivid, ddC), and Zidovudine (Retrovir, AZT, ZDV); and/or Protease Inhibitors (PIs) such as Amprenavir (Agenerase), Atazanavir (Reyataz), Fosamprenavir (Lexiva, 908), Indinavir (Crixivan), Lopinavir+Ritonavir (Kaletra), Nelfinavir (Viracept), Ritonavir (Norvir), Saquinavir (Fortovase, Invirase), and Tipranavir (Aptivus). Further, examples of suitable antibiotics include penicillin, tetracycline, chloramphenicol, minocycline, doxycycline, vancomycin, bacitracin, kanamycin, neomycin, gentamycin, erythromycin, geldanamycin, geldanamycin analogues and cephalosporins. Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

The compositions described herein may be prepared in any suitable manner. In some embodiments, the compositions may be prepared in a capsule. In such embodiments, the encapsulation process includes the feeding of components (e.g., Aloe vera extract, inulin and/or Fucose) in a generally powder form to an encapsulating machine to produce finished capsules.

For example, the encapsulation machine may comprise a powder chute that is loaded with the composition in powder form and a capsule chute that is loaded with capsules. A disc that fits capsules may be placed on a capsule dosing track. The disc may be provided in two parts. The first part may hold a first portion of the capsule and a second part may hold a second portion of the capsule. The machine may then be actuated on to allow the disc to load the capsules. Once the capsules have been loaded, a material feed shoot may be properly placed and activated to fill the capsules with the desired composition. Once the capsules are filled, the material feed shoot may be removed and the disk containing the other portion of the capsules may be properly aligned. Once properly aligned pressure may be applied to close the capsules. The finished capsules may then be removed for packaging.

The compositions and methods as described above are believed to be effective in treating a variety of disease conditions. For example, as described in Example 1, a treatment including administering Aloe vera and inulin was shown to significantly reduce the pain of a patient suffering from RSD. In addition, as described in Example 2, a treatment including administering Aloe vera and inulin was shown to significantly reduce the symptoms of a patient suffering from Fibromyalgia. As another example, as described in Example 6, a treatment including administering Aloe vera and inulin was shown to significantly reduce the symptoms associated with Chrohn's Disease. In addition, as discussed in Examples 7-10, a treatment including administering Aloe vera and inulin was shown to increase the CD4 count of patients suffering from HIV/AIDS. Further, as discussed in the study of Example 12, a treatment including administering Aloe vera product derived from TPA was shown to increase the CD4 count of patients suffering from HIV/AIDS in combination with antiviral and antibiotic therapies.

### EXAMPLES

The following examples are presented for illustrative purposes and are not intended to limit the scope of the claims that follow.

### Example 1

### Treatment of RSD

30 year old white male injured when 400 lbs of windows fell onto his left arm. Pain from Left Shoulder down to the Left hand, with inability to move the extremity without extreme pain. Therefore unable to work, exercise, or use left arm, required to carry the arm in a sling. Previous MRI, CT and Electromyography confirmed the diagnosis of Reflex Sympathetic Dystrophy (RSD). RSD is a disorder of the extremities that is characterized by pain, swelling, limited range of motion, vasomotor instability, skin changes, and patchy bone demineralization; it frequently follows injury, surgery, or a vascular event. The pain response probably represents increased sensitivity of injured axons to epinephrine and other substances released by local sympathetic nerves. He received 8 months of Physical Therapy, TENS nerve block, and medications, without any improvement.

He was started on 400 mg of freeze-dried TPA plus 600 mg inulin powder (extracted from chicory roots) each day and began a weight loss program to eliminate the obesity due to inactivity. About five months after starting the TPA/inulin treatment the patient reported that the arm felt much different. About one year after starting the TPA/inulin treatment the physician noted that patient had less pain on left side, increased mobility without pain, and able to touch the left arm with much less sensitivity. He was now able to walk 2.5 miles slowly daily, with less ankle pain. He was able to exercise moderately now that the pain in the RSD-affected arm had improved. First time he had been with less pain in the 2 years following his accident.

### Example 2

### Treatment of Fibromyalgia

A 60 year old white female presents with multiple symptoms of Fibromyalgia, dizziness, vertigo, right ear pain, headaches, memory impairment, cardiac palpitations, irritable bowel, migratory myalgias and arthralgias, and progressive fatigue. She is a concert cellist who has found it more difficult to remember the scores in her music, and in fact has been unable to concentrate and perform her music. Diagnoses include Fibromyalgia, psoriasis, psoriatic arthritis, thyroid disease, and Meniere's disease.

Her psoriasis had been treated using Methotrexate (10 mg weekly); she was placed on a regimen of Guaifenesin (600 mg twice daily) to detoxify the body of phosphate buildup as seen with Fibromyalgia. She was also started on 150 mg freeze-dried TPA plus 4000 mg inulin powder (extracted from chicory roots) per day. She also was taking supplements containing glucosamine, chondroitin sulfate and methylsulfonylmethane.

She was on this regimen for about five and one-half months. She noted that the memory improved to the point of near perfect cello performances, less vertigo, improvement of the psoriasis with only 5 mg per week Methotrexate, and only 1-2 headaches per month compared to 1-2 headaches per week earlier.

Her overall symptoms were 50-60% less with the above regimen, and she had reclaimed her life with improvement in all of her previous symptoms.

### Example 3

### Treatment of Hepatitis C

48 year old white female with 2 year history of Hepatitis C. Good health most of her life with 2 normal childbirths. Within the last 2 years she was found to have elevated liver function tests. Her labs revealed an SGOT of 94, SGPT of 116 with Hepatitis C, subtype 1A being diagnosed. Liver Biopsy revealed Portal Fibrosis, but no definite cirrhosis. Her Hepatitis Viral Load by PCR was 850,000. She refused Interferon due to the side effects and poor response rate. About five months later, her Hepatitis C Viral Load by PCR was 7,070,000. She was started on 6 capsules of Totaloe which contained 600 mg freeze-dried TPA along with 900 mgs inulin powder (extracted from chicory roots) per day. After 1 month of therapy, her Hepatitis C Viral Load by PCR had dropped to 5,650,000 viral particles. Her Hepatitis C Virus RNA Log was 6.8. Her SGOT was 32, and her SGPT was 26, both normal liver function tests as relates to her hepatitis.

### Example 4

### Treatment of Fibromyalgia

69 year old white female presents with typical Fibromyalgia symptoms, and also taking Asacol for Crohn's disease. Started with Guaifenesin for ditoxication of phosphate build up. She was placed on a regime of 200 mg freeze-dried Aloe vera (isolated from TPA) plus 300 mg inulin powder (extracted from chicory roots), and instructed to decrease the Asacol from 3 per day to 2 per day, and slowly titrated off of the Asacol, while increasing the freeze-dried Aloe vera isolated to 300 mg per day and the inulin powder to 450 mg per day over a period of 18 months.

She has had no flares of her Crohn's disease, and her Fibromyalgia has improved markedly. Her sleep is more restful, and the muscle aches and pains have also resolved greatly.

### Example 5

### Treatment of Hepatitis C

57 year-old white male, Vietnam prisoner of war who spent months in a septic pool, found to have Hepatitis C with a viral load of 2,350,000; he was unable to take any conventional Interferon treatment due to the cost. He was started on 600 mg of freeze-dried TPA isolate plus 900 mg inulin powder (extracted from chicory roots) per day. About three months later, a repeat Hepatitis C viral load was done to reveal a count of 1,160,000. No abnormal hepatic function.

### Example 6

### Treatment of Crohn's Disease

9 year-old white male diagnosed with Crohn's Disease following acute onset of abdominal pain, daily episodes of diarrhea with bleeding, weight loss, and sometimes dehydration. His Gastroenterologist had him taking a total of 17 pills daily, which included Methotrexate and Asacol. After 2 years of this regimen and his condition only worsening, the last option was surgery to remove his small intestine.

He initially started taking 125 mg per day freeze-dried TPA isolate plus 4872 mg per day inulin powder (extracted from chicory roots). Within 4 months his symptoms had improved to where the diarrhea was occasional and the bleeding was little to none. He had regained his lost weight and was no longer missing 2-3 days/week of school. His Gastroenterologist found that his colonoscopy had cleared of the hundreds of ulcerations within the colon, and over the next year was titrated off of the methotrexate as well as the Asacol.

### Examples 7-10

### Treatment of HIV/AIDS

A study was undertaken in with four HIV positive patients in the Panama City, Fla. area. The individual study participants are discussed in Examples 7-10. The initial results are as follows:

| Patient | Pre-treatment CD4/HIV PCR | Post-treatment CD4/HIV PCR |
|---|---|---|
| C. A. | 207 (25%)/<400 | 300 (29%)/<400 |
| R. C. | 172 (18%)/126,000 | 256 (22%)/89,000 |
| G. E. | 76 (8%)/<400 | 125 (11%)/<400 |
| W. S. | 150 (16%)/417,000 | 218 (19%)/156,000 |

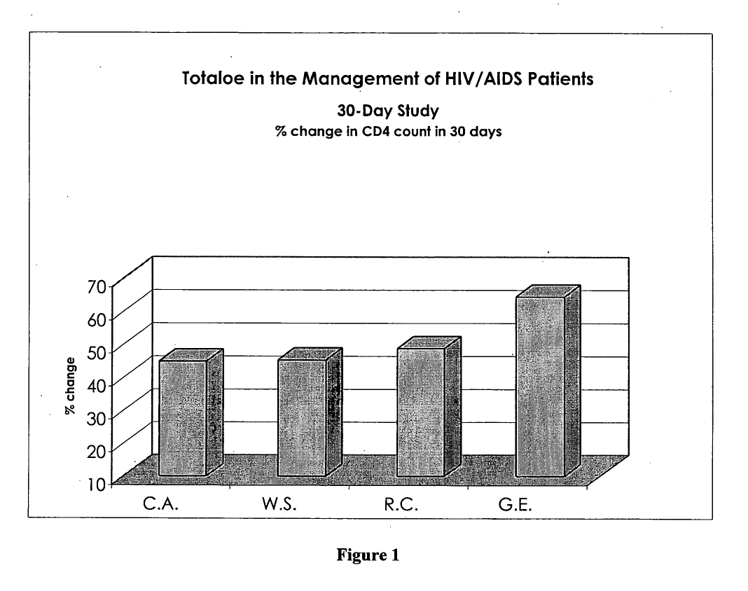

The study was only 4 weeks long. All of the patients had active HIV for an extended period of time. They were on standard therapy prior to this four weeks, and yet their CD4 lymphocyte count improved. In addition, the HIV PCR viral count dropped, or was already at a low level. Our Infectious Disease doctor stated that when the CD4 count reached 50% of the overall lymphocyte count, and the HIV PCR viral load became less than (<) 400, then the symptoms of AIDS would no longer be evident. The percentage change in the CD4 count of each of the participants during the study period are shown below in FIG. 1.

The only difference in the Pre-treatment and Post-treatment results was the addition of 6 capsules of Totaloe from Aluwe, LLC. Totaloe is a total process glyconutrient from the Aloe vera plant. The Aloe vera plant is processed in a very special manner to obtain very large molecules of long chain polysaccharides, which play an essential part in modulating the immune system.

### EXAMPLE 7

### Treatment of HIV/AIDS

CA is a 48-year-old white female with HIV disease for 7 years. Patient has never had any opportunistic infections although at one point in time she had some mild mental status changes that have been attributed to HIV dementia, which resolved with therapy and antiretrovirals. Patient is currently on Viread, Epivir, and Sustiva along with prophylactic Zithromax and Septra. Patient is also on over the counter Iron, Vitamin C and Vitamin E. She took 600 mg of freeze-dried full spectrum TPA plus 900 mg of inulin powder (from chicory root extract) daily during 30 days.

| | | |
|---|---|---|
| Pre-treatment CD4 | 207(25%) | HIV PCR <400 |
| Post-treatment CD4 | 300(29%) | HIV PCR <400 |

### Example 8

### Treatment of HIV/AIDS

RC is a 38-year-old white male with HIV disease for 14 years. Patient has multiple other medical problems including Insulin Dependent Diabetes Mellitus, Gastro esophageal Reflux Disease, Cerebrovascular Accident (stroke) and Hyperlipidemia. He has had no opportunistic infections. Patient is currently on Viread, Hivid, Kaletra, Invirase, Diflucan, Pepcid, Pamelor, Tegretol, Lipitor and Lantus Insulin. He took 600 mg of freeze-dried full spectrum TPA plus 900 mg of inulin powder (from chicory root extract) daily during 30 days.

| | | |
|---|---|---|
| Pre-treatment CD4 | 172(18%) | HIV PCR 126,000 |
| Post-treatment CD4 | 256(22%) | HIV PCR 89,000 |

### Example 9

### Treatment of HIV/AIDS

GE is a 43-year-old white male with HIV disease for 18 years. He has a history of HIV Wasting Syndrome with Cytomegalovirus retinitis and Pneumocystis carinii pneumonia. He is currently receiving Viread, Combivir, Kaletra, Oxandrin, Marinol, Valcyte, Prevacid and Lomotil as needed. He took 600 mg of freeze-dried full spectrum TPA plus 900 mg of inulin powder (from chicory root extract) daily during 30 days.

| | | |
|---|---|---|
| Pre-treatment CD4 | 76(08%) | HIV PCR <400 |
| Post-treatment CD4 | 125(11%) | HIV PCR <400 |

### Example 10

### Treatment of HIV/AIDS

WS is a 48-year-old white male with HIV for 8 years. He has a history of Syphilis, Hyperlipidemia, Depression, Migraine Headaches, Gastro esophageal Reflux Disease, Neuropathy and HIV Wasting Syndrome. He is currently taking Viread, Zerit, Videx EC, Neurontin, Zyrtec, Maxalt, Zantac, Zoloft, Lopid and Lortab for pain. He took 600 mg of freeze-dried full spectrum TPA plus 900 mg of inulin powder (from chicory root extract) daily during 30 days.

| | | |
|---|---|---|
| Pre-treatment CD4 | 150(16%) | HIV PCR 417,000 |
| Post-treatment CD4 | 218(19%) | HIV PCR 156,000 |

### Example 11

### Treatment of HTLV I/II

27 year-old white female presented with a positive Red Cross Blood Test for HTLV I/II. She took 600 mg of freeze-dried full spectrum TPA plus 900 mg of inulin powder (from chicory root extract) daily during 30 days. Also treated with Metformin for Polycystic Ovary Syndrome. At the end of the 30 days the patient returned for repeat blood test and final report shows no evidence of HTLV I/II. This T-cell lymphotrophic virus is associated with peripheral T-cell neoplasm of which Adult T cell lymphoma/leukemia is one type.

### Example 12

### Study of Treatment of HIV/AIDS by Tropical Diseases Research Centre (TDRC), Ndola, Zambia

The aim of this study was to investigate the benefits of TotAloe in the management of patients with HIV/AIDS and who are on ARVs in Zambia. TotAloe is a nutritional supplement manufactured by Aluwe International in the United States of America. This product has been on the international market for a long time. TotAloe is made from a process known as Total Process Aloe or TPA. The TPA method extracts up to four times more Aloe Vera solids than whole leaf processing. TPA carefully preserves the full range of beneficial nutrients found in the native plant-especially the immune modulating factors, known as long chain polysaccharides. It comes in a format called Veggie caps and includes Galactose, Xylose, Glucose, N-acetyl Glucosamine, Mannose, N-acetyl Galactosamine and N-acetyl Neuramic Acid (sialic acid). There are no preservatives, fillers, artificial colours or animal ingredients.

The objectives of the study were to:

1. Determine the impact of TotAloe on haematological, biochemical, and immunological markers in patients on ARVs on a monthly basis for three months.

2. Assess any change in the general condition of the patients on ARVs and taking TotAloe in terms of body weight, occurrences of opportunistic infection conditions such as oral thrush, diarrhea, and body rashes over a period of three months.

Methodology.

Study design: This was an observational study.

Study site: The study was conducted at TDRC clinic. Patients were recruited from the TDRC clinic and the ARV clinic at Ndola Central Hospital.

Study population: All patients attending the NCH ARV clinic and TDRC clinic and meeting the inclusion criteria below were eligible for enrolment into the study.

Inclusion Criteria:

1. Adult males/females aged 18 years and above.

2. Ambulatory.

3. Fully conscious.

4. With/without an AIDS defining symptom/sign.

5. Willing to give informed consent.

Exclusion Criteria:

1. Bedridden.

2. Mentally confused

3. Unable to take orally.

4. Not willing to give consent.

Sample Size: Twelve Patients were Enrolled into the Study

Details of the Fieldwork.

1. Screening:

Screening of potential study participants was done at TDRC clinic and NCH ARV clinic. The clinic doctors screened the participants. Eligible participants were then referred to the study doctor at TDRC clinic.

2. Enrolment:

The study doctor at TDRC clinic welcomed the potential study participants. He explained the objectives of the study. Thereafter he obtained a written Informed consent from the participants. History of the presenting complaints was recorded in the Case Record File (CRF). The doctor then physically examined the participant and findings were entered in the CRF. The study nurse measured the weight, blood pressure, and body temperature of the study participant. Venous blood was be drawn from the participant and was be analyzed for haematological, biochemical and immunological parameters. Thereafter the participant was issued with a month supply of nutritional supplement, TotAloe. TotAloe was supplied by Aluwe International LLC. The dosage was be explained to the participants. Participants were instructed to continue taking ARVs and any antibiotics prescribed to them by a doctor for any opportunistic infections. The participants were then given appointment dates at one month interval for three months.

3. Follow Up:

At one-month intervals for three months the study doctor reviewed participants. History of presenting complaints was recorded. The doctor reviewed complaints presented at the previous visit. The doctor conducted a physical examination of the participant. The nurse measured the body weight, blood pressure, and body temperature. Venous blood was again be drawn from the participants and analyzed for haematological, biochemical, and immunological parameters. Participants received a one-month supply of TotAloe.

Results:

i) Profile of the Study Patients:
a) The mean age of the patients was 39.9 years (range: 24-54 years).
b) Sex: There were 8 males and 4 females.
c) Address: 8 patients were Ndola residents while 4 were Luanshya (a nearby town) residents.
d) Duration on the Antiretroviral drugs before commencement of TotAloe: The mean duration was 12.5 months (range: 0-40 months)

ii) Evaluation of TotAloe.

All twelve study patients were reviewed at one-month interval for three months. The results were analyzed for the clinical and immunological impact (efficacy) and for the safety of TotAloe.

a) Clinical Impact:
i) Mortality: There was no recorded death in the study over the three months of the study duration.

ii) Improvement in General condition, body weight, and study patients complaints and resumption of normal life. The observations are summarized in Table 1.

TABLE 1
Summary of clinical impact of TotAloe.

| Parameter | Visit 1 | Visit 2 | Visit 3 | Visit 4 |
|---|---|---|---|---|
| 1. General | 5 | 9 | 11 | 12 |
| condition | | | | |
| (assessed as | | | | |
| being good) | | | | |
| 2. Body | | | | |
| weight (kg) | | | | |
| Mean: | 63.9 | 64.5 | 65.3 | 66.7 |
| Range: | 51.0-85 | 53-87 | 56-89 | 57-89 |
| 3. Complaints | 11 | 5 | 8 | 2 |
| (present) | | | | |

General Condition.

General condition was defined as the general appearance of the patient on physical examination. The study doctor scored it as good, fair, and bad. At Visit 1 only 5 study patients were scored as good general condition. At Visit 4 (three months on TotAloe) all the 12 study patients were scored as good general condition.

Body Weight.

The mean body weight for the 12 study patients at Visit 1 was 63.9 kg (range: 51.0-85.0 kg). This increased to 66.7 kg (range: 57-89 kg) at Visit 4. Thus the mean increase in body weight over a period of three months was 2.8 kg. The lowest minimum body weight rose from 51.0 kg at Visit 1 to 57.0 kg at Visit 4.

Complaints.

At Visit 1, 11 study patients had at least one complaint. At the end of the study (Visit 4) only 2 study patients had at least one complaint.

Resumption of Normal Life.

One patient who could not walk unaided at Visit 1 was able to walk unaided and had resumed work before the end of study (Visit 4). This patient used to move from place to place using a wheelchair. Two patients whose businesses had slowed down due to their ill-health were able to revamp their businesses before the study ended as a result of improved life. One evangelist whose practice had suffered as a result of illness had resumed his duties before the end of the study.

b) Immunological Impact:

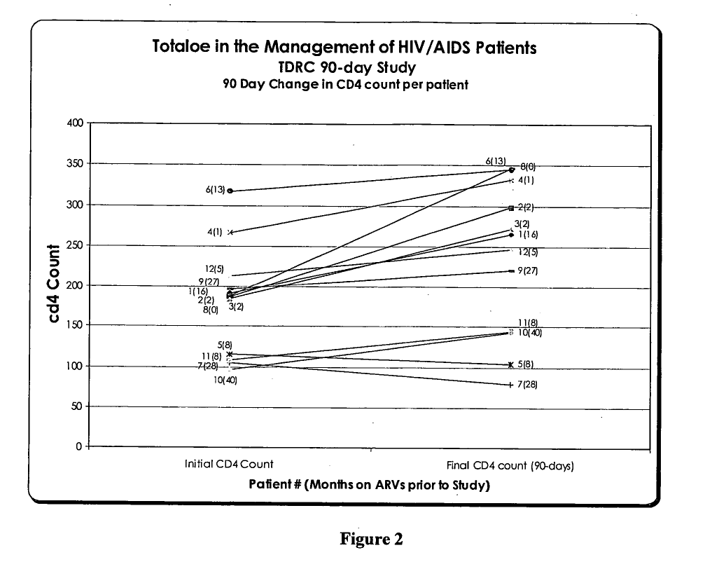

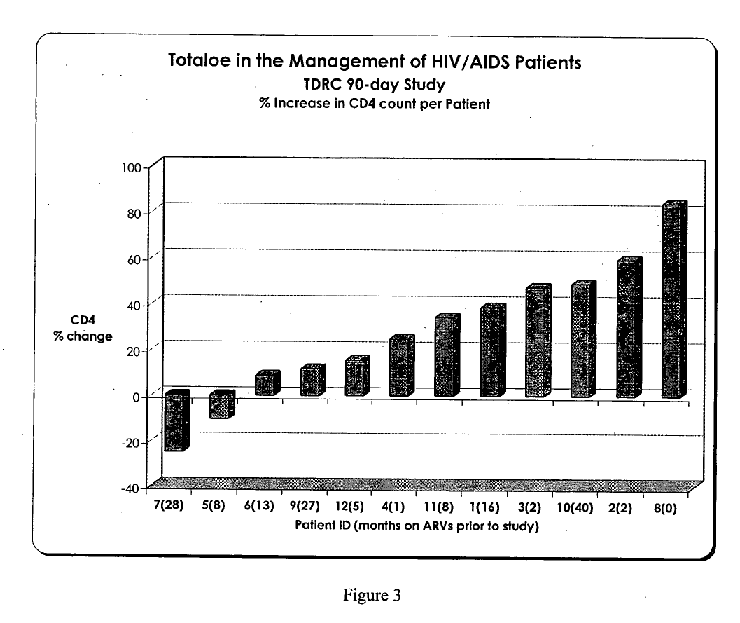

This was measured by the changes in CD4 count over a period of three months. The observations are summarized in Table 2. FIG. 2 shows a graph depicting the change in CD4 count by time and FIG. 3 shows a graph depicting the change in CD4 count by patient.

i) Mean CD4 count: This rose from 180 cells/μl at Visit 1 to 233 cells/μl at Visit 4.

ii) Individual increase in CD4 count: At one-month follow up CD4 count increased in 7 study patients while for the remaining 5 the count decreased. At one month follow up there was a dramatic rise in CD4 count in 5 patients ranging from 31.2 to 108.6%. At three-months follow up CD4 count had increased in 9 out of 11 study patients whose blood was analyzed for CD4 count when compared with CD4 at Visit 1.

iii) Percentage increase in CD4 count at follow up Visits as compared with Visit 1:

The Mean percentage increase in CD4 count rose from 23.3% at Visit 2 to 29.7% at Visit 4 when compared with CD4 count at Visit 1.

TABLE 2
Summary of immunological observations.

| | CD4 count at | CD4 count at | CD4 count at | CD4 count at | Percentage increase | Percentage increase | Percentage increase |
|---|---|---|---|---|---|---|---|
| ID | Visit 1 | Visit 2 | Visit 3 | visit 4 | in CD4 count at | in CD4 count at | in CD4 count at |
| number | (per μl) | (per μl) | (per μl) | (per μl) | Visit 2 (from Visit 1) | Visit 3 (from Visit 1) | Visit 4 (from Visit 1) |
| 1 | 191 | 162 | 225 | 265 | −15.1 | 17.8 | 38.7 |
| 2 | 187 | 297 | 304 | 298 | 58.8 | 63.1 | 59.4 |
| 3 | 184 | 186 | 250 | 271 | 1.1 | 35.9 | 47.3 |
| 4 | 266 | 349 | 237 | 332 | 31.2 | −10.9 | 24.8 |
| 5 | 116 | 242 | 98 | 104 | 108.6 | −17.2 | −10.3 |
| 6 | 317 | 268 | 286 | 345 | −15.5 | −9.8 | 8.8 |
| 7 | 105 | 79 | 128 | 79 | −24.8 | 21.9 | −24.8 |
| 8 | 188 | 390 | 365 | 346 | 107.4 | 94.1 | 84.0 |
| 9 | 197 | 337 | 220 | — | 71.1 | 11.7 | — |
| 10 | 96 | 105 | 103 | 143 | 9.4 | 7.2 | 48.9 |
| 11 | 108 | 94 | 173 | 145 | −13.0 | 60.2 | 34.2 |
| 12 | 213 | 155 | 153 | 246 | −27.2 | −28.2 | 15.5 |
| Mean | 180 | 222 | 211 | 233 | 23.3 | 20.5 | 29.7 |
| Range: | 96-317 | 79-390 | 98-365 | 79-346 | −27.2-108.6 | −28.2-94.1 | −24.8-84.0 |

c) Safety of TotAloe.

This was determined by presence of side effects and state of laboratory parameters (Biochemical and haematological) results.

i) Side effects: There were no reported side effects to TotAloe such as body rashes, headache, vomiting, nausea, loss of sleep, and loss of appetite in all the study patients.

ii) Biochemical parameters: The observations here are summarized in Table 3.

iii) Haematological parameters: The observations here are summarized in Table 4.

TABLE 3
Summary of biochemical results at all the first three Visits.

| Parameter | Visit 1 | Visit 2 | Visit 3 |
|---|---|---|---|
| 1. Creatinine: | | | |
| Mean | 82.0 | 85.3 | 87.8 |
| Range | 65-122 | 63.0-101.0 | 75.0-101.0 |
| 2. Random | | | |
| Blood sugar: | | | |
| Mean | 4.0 | 5.8 | 4.6 |
| Range | 2.5-5.5 | 3.6-6.7 | 3.6-6.2 |
| 3. GOT: | | | |
| Mean | 25.1 | 30.6 | 31.6 |
| Range | 12.0-78.0 | 16.0-59.0 | 17.0-84.0 |
| 4. Total | | | |
| Proteins: | | | |
| Mean | 95.5 | 93.9 | 80.4 |
| Range | 36.6-112.8 | 62.1-122.5 | 34.1-106.0 |

As can be seen TotAloe had no adverse effects on all the four biochemical parameters measured in the study patients.

iii) Haematological Parameters:

TABLE 4
Summary of haematological results at all the first three Visits.

| | Visit 1 | Visit 2 | Visit 3 |
|---|---|---|---|
| Parameter | (g/dl) | (g/dl) | (g/dl) |
| 1. Haemoglobin | | | |
| Mean: | 11.8 | 11.5 | 12.4 |
| Range: | 8.2-14.9 | 6.9-15.2 | 8.6-15.1 |
| 2. White blood | | | |
| cells | | | |
| Mean: | 5.5 | 4.8 | 5.5 |
| Range: | 2.6-8.7 | 2.6-6.7 | 3.4-7.5 |
| 3. Red blood cells | | | |
| Mean: | 3.7 | 3.6 | 3.7 |
| Range: | 2.8-4.5 | 3.2-4.5 | 3.0-4.6 |
| 4. ESR | | | |
| Mean: | 85.3 | 95.8 | 57.3 |
| Range: | 37.0-130 | 70.0-122.0 | 10-105 |

The observations from Table 4 show that there were no adverse effect of TotAloe on blood cell formation and distribution.

### CONCLUSIONS

Only twelve patients were observed in this study and the duration of the observation was only three months. This means that the conclusions from this study have to be treated with caution like any other new product. Very convincing observations would have been observed if the sample size was larger and the duration of the observation longer.

However, safety results from this study demonstrated that TotAloe as a nutritional supplement and immune booster is safe over a period of three months. From the efficacy view, the combination of ARVs and TotAloe seemed to improve significantly the clinical well being of the majority of the patients over a very short time (less than two months on TotAloe). This observation was very marked in three patients who were able to resume work just after being on TotAloe-ARVs combination for only two months despite being on ARVs alone for a good number of months. Immunological markers such as CD4 count also increased tremendously in the majority of patients over a short period of three months after taking TotAloe-ARVs combination. In some instances CD4 count more than doubled over a period of only one month on TotAloe-ARV combination.

TotAloe seems to work synergistically with ARVs in improving the clinical well being and immunological status of study patient. Because of the dramatic improvement in both clinical well being and immunological status observed in certain patients over only a period of three months, it is most possible that even on its own TotAloe might be an immune booster enough to function as effective as ARVs in patients who may not want to be on ARVs.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations, which fall within the spirit and broad scope of the invention.

What is claimed is:

1. A composition useful for treating one or more disease conditions in a mammal comprising an Aloe vera isolate derived from total process aloe and a prebiotic.

2. The composition of claim 1, wherein the disease condition is one or more selected from the group consisting of a neurological syndrome, a chronic pain illness, an inflammatory bowel disease, and a viral disease.

3. The composition of claim 2, wherein the neurological syndrome includes reflex sympathetic dystrophy, the chronic pain illness includes fibromyalgia, the inflammatory bowel disease includes Crohn's disease, and the viral disease includes HIV/AIDS.

4. The composition of claim 1, wherein the disease condition includes HIV/AIDS.

5. The composition of claim 1, wherein the prebiotic comprises a fructan.

6. The composition of claim 5, wherein the fructan is an inulin-type fructan derived from a chicory root.

7. The composition of claim 1, further comprising one or more pharmaceutically acceptable excipients, carriers, diluents, or adjuvants.

8. The composition of claim 1, wherein the mammal is a human.

9. The composition of claim 1, wherein the composition is in a pharmaceutically acceptable dosage forms further comprising one or more pharmaceutically acceptable excipients, carriers, diluents or adjuvants.

10. The composition of claim 9, the dosage forms being capable of releasing the active ingredients in the gut of the mammal.

11. The composition of claim 1, wherein the composition is administered along with other therapeutic regimens.

12. The composition of claim 11, wherein the other therapeutic regimens comprise antiviral drug therapy regimens.

13. The composition of claim 1, wherein the composition is administered orally.

14. The composition of claim 1, wherein the composition is administered in an encapsulated powder.

15. The composition of claim 1, wherein the composition is administered as a reconstituted liquid.

16. The composition of claim 1, wherein the Aloe vera is about 30-70% of the composition by weight.

17. The composition of claim 1, wherein the prebiotic is about 30-70% of the composition by weight.

18. The composition of claim 1, further comprising Fucose.

19. The composition of claim 18, wherein the Fucose is L-Fucose.

20. The composition of claim 18, wherein the Aloe vera is about 30-70% of the composition by weight.

21. The composition of claim 18, wherein the prebiotic is about 30-70% of the composition by weight.

22. The composition of claim 18, wherein the Fucose is about 10-50% of the composition by weight.

23. A composition useful for treating HIV/AIDS comprising an Aloe vera isolate, a prebiotic, and Fucose.

24. The composition of claim 23, wherein the prebiotic comprises a fructan.

25. The composition of claim 24, wherein the fructan is an inulin-type fructan derived from a chicory root.

26. A method to treat a disease condition, comprising delivering a composition containing an Aloe vera isolate derived from total process aloe and a prebiotic to the gut of a mammal in need thereof.

27. The method of claim 26, wherein the disease condition is one or more selected from the group consisting of a neurological syndrome, a chronic pain illness, an inflammatory bowel disease, and a viral disease.

28. The method of claim 27, wherein the neurological syndrome includes reflex sympathetic dystrophy, the chronic pain illness includes fibromyalgia, the inflammatory bowel disease includes Crohn's disease, and the viral disease includes HIV/AIDS.

29. The method of claim 26, wherein the prebiotic comprises a fructan.

30. The method of claim 29, wherein the fructan is an inulin-type fructan derived from a chicory root.

31. A method to treat a disease condition, comprising delivering a composition containing an Aloe vera isolate derived from total process aloe to the gut of a mammal in need thereof along with other therapeutic regimens.

32. The method of claim 31, wherein the disease condition is one or more selected from the group consisting of a neurological syndrome, a chronic pain illness, an inflammatory bowel disease, and a viral disease.

33. The method of claim 32, wherein the neurological syndrome includes reflex sympathetic dystrophy, the chronic pain illness includes fibromyalgia, the inflammatory bowel disease includes Crohn's disease, and the viral disease includes HIV/AIDS.

34. The method of claim 31, wherein the prebiotic comprises an inulin-type fructan derived from a chicory root.

35. The method of claim 31, wherein the other therapeutic regimens comprise delivering an antiviral drug therapy regimen.
